SEQUENCE LISTING (2 sequences)

SEQ ID NO: 1 - DNA, 624 bp, human
atggactcggacgagaccgggttcgagcactcaggactgtgggtttctgtgctggctggtctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc

SEQ ID NO: 2 - protein, 208 aa, homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

(19) United States
(12) Patent Application Publication
(10) Pub. No.: US 2004/0259780 A1
(43) Pub. Date: Dec. 23, 2004

(54) Method for treating diabetes and obesity

(76) Inventors: Andrew Lawrence Glasebrook, Zionville, IN (US); Lisa Janine Hammond, Indianapolis, IN (US); Alexei Kharitonenkov, Carmel, IN (US); Tatiyana Shiyanova, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/483,283

(22) PCT Filed: Jul. 22, 2002
(86) PCT No.: PCT/US02/21290

Related U.S. Application Data
(60) Provisional application No. 60/308,702, filed on Jul. 30, 2001.
(60) Provisional application No. 60/347,991, filed on Jan. 10, 2002.

(51) Int. Cl. A61K 38/18

(52) U.S. Cl. 514/12

(57) ABSTRACT

A method for treating diabetes and obesity comprising administering an effective amount of fibroblast growth factor 21.

The present invention relates to methods for treating mammals suffering from non-insulin dependent Diabetes Mellitus (NIDDM: Type 2), insulin dependent diabetes (Type 1), as well as obesity, inadequate glucose clearance, hyperglycemia, hyperinsulinemia, and the like.

Diabetes mellitus is characterized in two broad groups based on clinical manifestations, namely, the non-insulin-dependent or maturity onset form, also known as Type 2; and the insulin-dependent or juvenile onset form, also known as Type 1. Clinically, the majority of Type 2, maturity onset diabetics are obese, with manifestations of clinical symptoms of the disease usually appearing at an age over 40. In contrast, Type 1, juvenile onset patients are not over-weight relative to their age and height, with rapid onset of the disease at an early age, often before 30, although Type 1 diabetes can occur at any age.

Diabetes mellitus is a metabolic disorder in humans with a prevalence of approximately one percent in the general population, with one-fourth of these being the Type 1 (Foster, D. W., Harrison's Principles of Internal Medicine, Chap. 114, pp. 661-678, 10th Ed., McGraw-Hill, New York). The disease manifests itself as a series of hormone-induced metabolic abnormalities that eventually lead to serious, long-term and debilitating complications involving several organ systems including the eyes, kidneys, nerves, and blood vessels. Pathologically, the disease is characterized by lesions of the basement membranes, demonstrable under electron microscopy.

Non-insulin-dependent Diabetes Mellitus (NIDDM: Type 2) is a debilitating disease characterized by high-circulating blood glucose, insulin and corticosteroid levels. The incidence of Type 2 diabetes is high and rising and is becoming a leading cause of mortality, morbidity and healthcare expenditure throughout the world (Amos et al., Diabetic Med. 14:S1-85, 1997).

The causes of Type 2 diabetes are not well understood. It is thought that both resistance of target tissues to the action of insulin and decreased insulin secretion (“β-cell failure”) occur. Major insulin-responsive tissues for glucose homeostasis are liver, in which insulin stimulates glycogen synthesis and inhibits gluconeogenesis; muscle, in which insulin stimulates glucose uptake and glycogen stimulates glucose uptake and inhibits lipolysis. Thus, as a consequence of the diabetic condition, there are elevated levels of glucose in the blood, and prolonged high blood sugar which is indicative of a condition which will cause blood vessel and nerve damage.

Currently, there are various pharmacological approaches for the treatment of Type 2 diabetes (Scheen et al., Diabetes Care, 22(9):1568-1577, 1999). They act via different modes of action: 1) sulfonyulureas essentially stimulate insulin secretion; 2) biguanides (metformin) act by promoting glucose utilization, reducing hepatic glucose production and diminishing intestinal glucose output; 3) α-glucosidase inhibitors (acarbose, miglitol) slow down carbohydrate digestion and consequently absorption from the gut and reduce postprandial hyperglycemia; 4) thiazol-idinediones (troglitazone) enhance insulin action, thus promoting glucose utilization in peripheral tissues; and 5) insulin stimulates tissue glucose utilization and inhibits hepatic glucose output. The above mentioned pharmacological approaches may be utilized individually or in combination therapy. However, each approach has its limitations and adverse effects.

Obesity is a chronic disease that is highly prevalent in modern society and is associated not only with a social stigma, but also with decreased life span and numerous medical problems including adverse psychological development, dermatological disorders such as infections, varicose veins, exercise intolerance, diabetes mellitus, insulin resistance, hypertension, hypercholesterolemia, and coronary heart disease. Rissanen et al., British Medical Journal, 301: 835-837 (1990).

Existing therapies for obesity include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery. Jung and Chong, Clinical Endocrinology, 35: 11-20 (1991); Bray, Am. J. Clin. Nutr., 55: 538S-544S (1992).

Considering the high prevalence of obesity in our society and the serious consequences associated therewith as discussed above, any therapeutic drug potentially useful in reducing weight of obese persons could have a profound beneficial effect on their health. There is a need in the art for a drug that will reduce total body weight of obese subjects toward their ideal body weight without significant adverse side effects and that will help the obese subject maintain the reduced weight level.

It is therefore desirable to provide a treatment regimen that is useful in returning the body weight of obese subjects toward a normal, ideal body weight. It is further desirable to provide a therapy for obesity that results in maintenance of the lowered body weight for an extended period of time.

Obesity is highly correlated with insulin resistance and diabetes in experimental animals and humans. Indeed, obesity and insulin resistance, the latter of which is generally accompanied by hyperinsulinemia or hyperglycemia, or both, are hallmarks of Type 2 diabetes. In addition, Type 2 diabetes is associated with a two- to fourfold risk of coronary artery disease. Despite decades of research on these serious health problems, the etiology of obesity and insulin resistance is unknown.

Type 1diabetics characteristically show very low or immeasurable plasma insulin with elevated glucagon. Regardless of what the exact etiology is, most Type 1patients have circulating antibodies directed against their own pancreatic cells including antibodies to insulin, to islet of Langerhans cell cytoplasm and to the enzyme glutamic acid decarboxylase. An immune response specifically directed against beta cells (insulin producing cells) leads to Type 1 diabetes. This specificity is supported by the above clinical picture, since beta cells secrete insulin while alpha cells secrete glucagon.

Current therapeutic regimens for Type 1 diabetes include modifications to the diet in order to minimize hyper-glycemia resulting from the lack of natural insulin, which in turn, is the result of damaged beta cells. Diet is also modified with regard to insulin administration to counter the hypoglycemic effects of the hormone. Whatever the form of treatment, parenteral administration of insulin is required for all Type 1 diabetics, hence the term “insulin-dependent” diabetes.

Thus, there is a need for an effective therapy of Type 2 diabetes that has fewer adverse effects than the available pharmaceutical approaches. Moreover, an effective alternative therapy to insulin could be useful for the treatment of Type 1 diabetes. The present invention provides a pharmacological therapy which stimulates glucose uptake and enhances insulin sensitivity in peripheral tissues and has fewer adverse effects than current treatment regimens for Type 2 diabetes. In addition, the present invention provides an alternative treatment for Type 1 diabetes. Furthermore, the present invention is useful for treating obesity by increasing energy expenditure by faster and more efficient glucose utilization.

The present invention provides a method for treating a mammal exhibiting one or more of Type 1 diabetes, Type 2 diabetes, obesity, insulin resistance, hyperinsulinemia, glucose intolerance, or hyperglycemia, comprising administering to said mammal in need of such treatment a therapeutically effective amount of FGF 21 having at least about 95% amino acid sequence identity to the amino acid sequence shown in SEQ ID NO:2.

The present invention further provides that said method of treating is sufficient to achieve in said mammal at least one of the following modifications: reduction in body fat stores, decrease in insulin resistance, reduction of hyperinsulinemia, increase in glucose tolerance, and reduction of hyperglycemia.

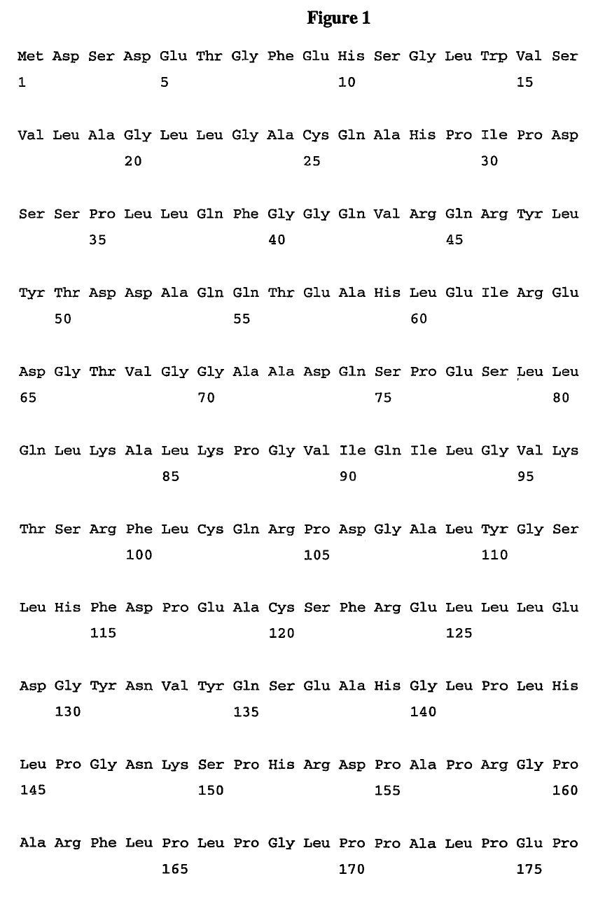

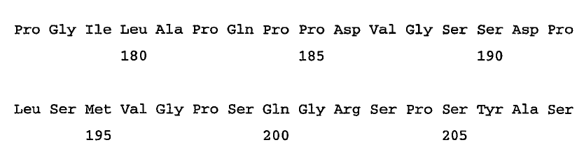

FIG. 1 shows the 208 amino acid sequence of fibroblast growth factor 21.

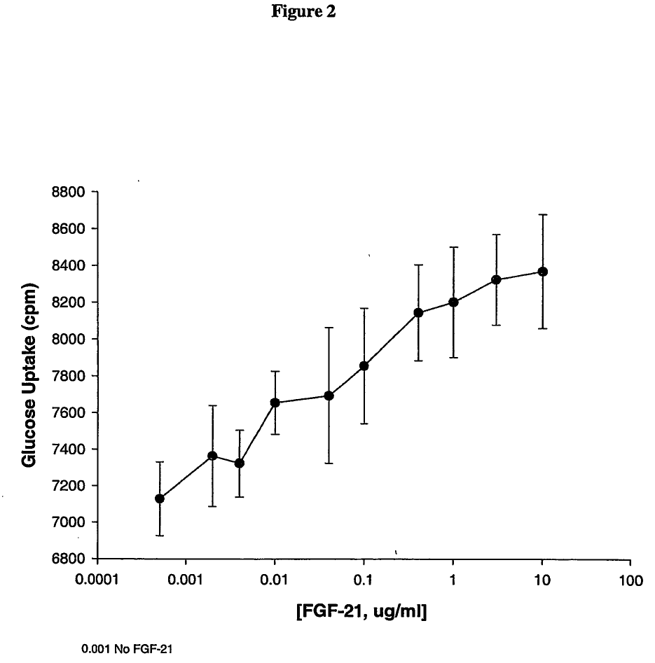

FIG. 2 shows FGF-21 stimulation of glucose uptake in 3T3-L1 adipocytes in a concentration dependent manner, performed at a sub-optimal concentration of insulin (5 nM).

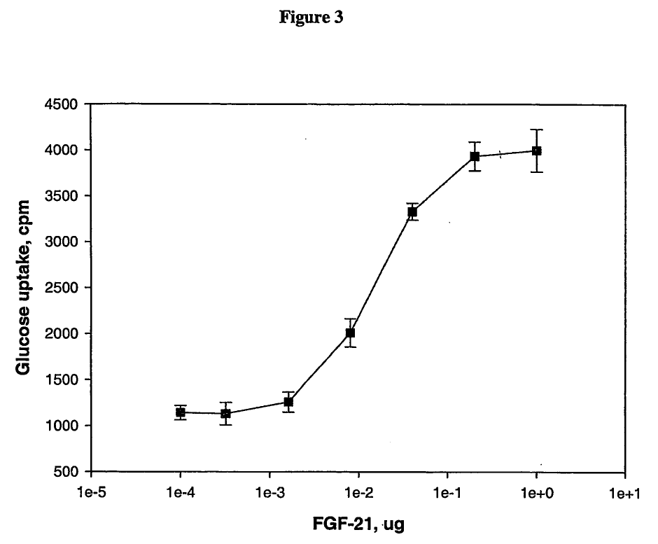

FIG. 3 shows FGF-21 stimulation of glucose uptake in 3T3-L1 adipocytes in a concentration dependent manner, in the absence of insulin.

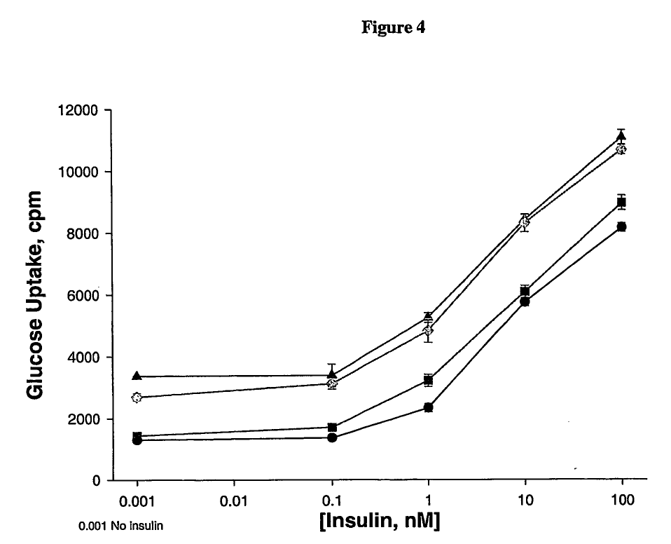

FIG. 4 shows FGF-21 stimulation of glucose uptake in 3T3-L1 adipocytes upon acute or chronic pretreatment in the presence of insulin. ●Control; ▪FGF-21 (1 μg/ml), acute pretreatment (20 minutes); ▴FGF-21 (1 μg/ml), chronic pretreatment (72 hours); [symbol] FGF-21 (1 μg/ml), chronic pretreatment (72 hours)+acute pretreatment (20 minutes).

For purposes of the present invention, as disclosed and claimed herein, the following terms are as defined below.

Fibroblast growth factor 21 (FGF-21) is a 208 amino acid polypeptide as shown in FIG. 1 and SEQ ID NO:2 and encoded by the DNA sequence indicated by SEQ ID NO:1.

Glucose intolerance can be defined as an exceptional sensitivity to glucose.

Hyperglycemia is defined as an excess of sugar (glucose) in the blood.

Hyperinsulinemia is defined as a higher-than-normal level of insulin in the blood.

Insulin resistance is defined as a state in which a normal amount of insulin produces a subnormal biologic response.

Obesity, in terms of the human subject, can be defined as that body weight over 20 percent above the ideal body weight for a given population (R. H. Williams, Textbook of Endocrinology, 1974, p.904-916).

The term “mature FGF polypeptide” refers to a polypeptide lacking a leader sequence and may also include other modifications of a polypeptide such as proteolytic processing of the amino terminus (with or without a leader sequence) and/or the carboxy terminus or other post-translational modifications understood by those with skill in the art.

A “therapeutically-effective amount” is the minimal amount of an active agent necessary to impart therapeutic benefit to a mammal. For example, a “therapeutically-effective amount” to a mammal suffering or prone to suffer or to prevent it from suffering from Type 2 diabetes or obesity is such an amount which induces, ameliorates or otherwise causes an improvement in the pathological symptoms, disease progression, physiological conditions associated with or resistance to succumbing to the afore described disorders.

Type 2 diabetes is characterized by excess glucose production in spite of the availability of insulin, and circulating glucose levels remain excessively high as a result of inadequate glucose clearance.

The present invention relates to a method of treating Type 2 diabetes comprising administering to a patient in need thereof an effective amount of fibroblast growth factor 21 (FGF-21) or variant thereof, a 208 amino acid polypeptide shown in FIG. 1.

In another aspect, the present invention relates to a method of preventing Type 2 diabetes comprising administering to a patient an effective amount of FGF-21 or variant thereof.

In another aspect, the present invention relates to a method of treating Type 1 diabetes comprising administering to a patient an effective amount of FGF-21 or variant thereof.

In another aspect, the present invention relates to a method of treating obesity comprising administering to a patient in need thereof an effective amount of FGF-21 or variant thereof.

In another aspect, the present invention relates to a method of treating a domestic animal e.g. cattle, pigs, sheep, horses, and the like, comprising administering an effective amount of FGF-21 or variant thereof, in order to reduce body fat stores. The reduction of body fat stores on a long term, or permanent basis in domestic animals would obviously be of considerable economic benefit to man, particularly since animals supply a major portion of man's diet; and the animal fat may end up as de novo fat deposits in man.

Fibroblast growth factors are large polypeptides widely expressed in developing and adult tissues (Baird et al., Cancer Cells, 3:239-243, 1991) and play crucial roles in multiple physiological functions including angiogenesis, mitogenesis, pattern formation, cellular differentiation, metabolic regulation and repair of tissue injury (McKeehan et al., Prog. Nucleic Acid Res. Mol. Biol. 59:135-176, 1998). Fibroblast growth factor 21 (FGF-21) is a recently identified FGF which has been reported to be preferentially expressed in the liver (Nishimura et al., Biochimica et Biophysica Acta, 1492:203-206, 2000; WO01/36640; and WO01/18172) and described as a treatment for ischemic vascular disease, wound healing, and diseases associated with loss of pulmonary, bronchia or alvelor cells or function and numerous other disorders. FGF-21 of the present invention is expressed primarily in liver, kidney, and muscle tissue, Example 2.

“FGF-21 variant” is intended to refer to an “active” or mature FGF-21, wherein activity is as defined herein, having at least about 90% amino acid sequence identity with an FGF-21 having a deduced amino acid sequences as shown in SEQ ID NO:2. Such FGF-21 variants include, for instance, wherein one or more amino acid residues are added, substituted or deleted, at the N- or C-terminus or within the sequences shown. Ordinarily, an FGF-21 variant will have at least about 90% amino acid sequence identity, preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% amino acid sequence identity with the amino acid sequence described, with or without the signal peptide.

“Percent (%) amino acid sequence identity” with respect to the FGF-21 amino acid sequences identified herein is defined as the percentage of amino acid residues in a candidate sequence that are identical with the amino acid residues in a FGF-21 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the % identity values used herein are generated using WU-BLAST-2 [Altschul et al., Methods in Enzymology 266: 460-480 (1996)]. Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a % amino acid sequence identity value is determined by dividing (a) the number of matching identical amino acid residues between the amino acid sequence of the FGF-21 of interest and the comparison amino acid sequence of interest (i.e., the sequence against which the FGF-21 of interest is being compared) as determined by WU-BLAST-2, by (b) the total number of amino acid residues of the FGF-21 of interest, respectively.

Included within the scope of this invention is the native FGF-21-signal sequence joined to an FGF-21 coding region and a heterologous signal sequence joined to an FGF-21 coding region. The heterologous signal sequence selected should be one that is recognized and processed, i.e., cleaved by a signal peptidase, by the host cell. A method of treating a condition or disorder with the FGF-21 of the present invention is meant to imply treating with FGF-21 with or without a signal peptide.

An “FGF-21 variant polynucleotide”, “FGF-21 polynucleotide” variant, or “FGF-21 variant nucleic acid sequence” are intended to refer to a nucleic acid molecule as defined below having at least about 80% nucleic acid sequence identity with the polynucleotide sequence as shown in SEQ ID NO: 1. Ordinarily, an FGF-21 polynucleotide variant will have at least about 90% nucleic acid sequence identity, yet more preferably at least about 91% nucleic acid sequence identity, yet more preferably at least about 92%, 93%, 94%, 95%, 96%, 97%, 98%, and most preferably at least about 99% nucleic acid sequence identity with the SEQ ID NO:1.

“Percent (%) nucleic acid sequence identity” with respect to the FGF-21 polynucleotide sequences identified herein is defined as the percentage of nucleotides in a candidate sequence that are identical with the nucleotides in the FGF-21 sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, % nucleic acid identity values are generated using the WU-BLAST-2 (BlastN module) program (Altschul et al., Methods in Enzymology 266: 460-480 (1996)). Most of the WU-BLAST-2 search parameters are set to the default values. Those not set default values, i.e., the adjustable parameters, are set with the following values: overlap span=1; overlap fraction=0.125; word threshold (T)=11; and scoring matrix=BLOSUM62. For purposes herein, a % nucleic acid sequence identity value is determined by dividing (a) the number of matching identical nucleotides between the nucleic acid sequence of the FGF-21-encoding nucleic acid molecule of interest and the comparison nucleic acid molecule of interest (i.e., the sequence against which the FGF-21-encoding nucleic acid molecule of interest is being compared) as determined by WU-BLAST-2, by (b) the total number of nucleotides of the FGF-21-encoding nucleic acid molecule of interest.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basis side chains (e.g., lysine, arginine, histidine), acidic side chains (e.g., aspartic acid, glutamic acid), uncharged polar side chains (e.g., glycine, asparagines, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in FGF-21 is replaced with another amino acid residue from the same side chain family. Alternatively, in another embodiment, mutations can be introduced randomly along all or part of a FGF-21 coding sequence, such as by saturation mutagenesis, and the resultant mutants can be screened for FGF-21 biological activity to identify mutants that retain activity. Following mutagenesis of SEQ ID NO:1, the encoded protein can be expressed by any recombinant technology known in the art and the activity of the protein can be determined.

We have discovered that FGF-21 stimulates glucose uptake and enhances insulin sensitivity in 3T3-L1 adipocytes, an in vitro model utilized for the study of adipose tissue metabolism, Example 3. FGF-21 is shown to stimulate glucose uptake in 3T3-L1 adipocytes in a concentration dependent manner at a sub-optimal concentration of insulin (5 nM), FIG. 2, and in the absence of insulin, FIG. 3. Additionally, FGF-21 induces glucose uptake in an ex vivo tissue model, Example 4.

A characteristic of Type 2 diabetes is the deficiency of glucose uptake in various tissue types including adipose tissue. Thus, FGF-21 is useful for treating Type 2 diabetes by lowering blood glucose levels. Moreover, FGF-21 is useful for treating obesity by increasing energy expenditure by faster and more efficient glucose utilization. Additionally, FGF-21 stimulates glucose uptake in 3T3-L1 adipocytes in an insulin independent manner (FIG. 3), indicating that it is useful for treating Type 1 diabetes as well.

FGF-21 is effective in animal models of diabetes and obesity, Examples 5 and 6. As metabolic profiles differ among various animal models of obesity and diabetes, analysis of multiple models was undertaken to separate the effects of hyperinsulinemia, hyperglycemia and obesity. The diabetes (db/db) and obese (ob/ob) mice are characterized by massive obesity, hyperphagia, variable hyperglycemia, insulin resistance, hyperinsulinemia and impaired thermogenesis (Coleman, Diabetes 31:1, 1982; E. Shafrir, in Diabetes Mellitus; H. Rifkin and D. Porte, Jr. Eds. (Elsevier Science Publishing Co., Inc., New York, ed. 4, 1990), pp. 299-340). However, diabetes is much more severe in the db/db model (Coleman, Diabetes 31:1, 1982; E. Shafrir, in Diabetes Mellitus; H. Rifkin and D. Porte, Jr. Eds. (Elsevier Science Publishing Co., Inc., New York, ed. 4, 1990), pp. 299-340). Zucker (fa/fa) rats are severely obese, hyperinsulinemic, and insulin resistant (Coleman, Diabetes 31:1, 1982; E. Shafrir, in Diabetes Mellitus; H. Rifkin and D. Porte, Jr. Eds. (Elsevier Science Publishing Co., Inc., New York, ed. 4, 1990), pp. 299- 340), and the fa/fa mutation may be the rat equivalent of the murine db mutation (Friedman et al., Cell 69:217-220, 1992; Truett et al., Proc. Natl. Acad. Sci. USA 88:7806, 1991). Tubby (tub/tub) mice are characterized by obesity, moderate insulin resistance and hyperinsulinemia without significant hyperglycemia (Coleman et al., J. Heredity 81:424, 1990).

The monosodium glutamate (MSG) model for chemically-induced obesity (Olney, Science 164:719, 1969; Cameron et al., Cli. Exp. Pharmacol. Physiol. 5:41, 1978), in which obesity is less severe than in the genetic models and develops without hyperphagia, hyperinsulinemia and insulin resistance, may also be examined. Finally, the streptozotocin (STZ) model for chemically-induced diabetes may be tested to examine the effects of hyperglycemia in the absence of obesity. STZ- treated animals are deficient in insulin and severely hyperglycemic (Coleman, Diabetes 31:1, 1982; E. Shafrir, in Diabetes Mellitus; H. Rifkin and D. Porte, Jr. Eds. (Elsevier Science Publishing Co., Inc., New York, ed. 4, 1990), pp. 299-340).

The FGF-21 administered according to this invention may be generated and/or isolated by any means known in the art such as described in Sambrook et al., Molecular Cloning: A Laboratory Manual, Cold Spring Harbor Laboratory Press, NY (1989).

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for FGF-21.

The FGF-21 used in the treatment of Type 1 diabetes, Type 2 diabetes or obesity can be formulated according to known methods to prepare pharmaceutically useful compositions. A desired formulation would be one that is a stable lyophilized product or aqueous solution of high purity with optional pharmaceutically acceptable carriers, excipients or stabilizers [Remington's Pharmaceutical Sciences 16th edition (1980)].

FGF-21 may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition (1980).

Sustained-release preparations may be prepared. Suitable examples of sustained-release preparations include semipermeable matrices of solid hydrophobic polymers containing the therapeutic agent(s), which matrices are in the form of shaped articles, e.g., films, or microcapsules. Examples of sustained-release matrices include polyesters, hydrogels [for example, poly(2-hydroxyethyl-methacrylate), or poly(vinylalcohol)], polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. Microencapsulation of recombinant proteins for sustained release has been successfully performed with human growth hormone (rhGH), interferon, and interleukin-2. Johnson et al., Nat. Med. 2(7): 795-9 (1996); Yasuda et al., Biomed. Ther. 27: 1221-3 (1993); Hora et al., Bio/Technology 8(8): 755-8 (1990); Cleland, “Design and Production of Single Immunization Vaccines Using Polylactide Polyglycolide Microsphere Systems” in Vaccine Design: The Subunit and Adjuvant Approach, Powell and Newman, Eds., Plenum Press, NY, 1995, pp. 439-462 WO 97/03692; WO 96/40072; WO 96/07399; and U.S. Pat. No. 5,654,010.

The sustained-release formulations of these proteins may be developed using polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. See Lewis, “Controlled release of bioactive agents from lactide/glycolide polymer” in Biodegradable Polymers as Drug Delivery Systems (Marcel Dekker; New York, 1990), M. Chasin and R. Langer ads.) pp. 1-41.

While polymers such as ethylene-vinyl acetate and lactic acid-glycolic acid enable release of molecules for over 100 days, certain hydrogels release proteins for shorter time periods.

It is contemplated that FGF-21 may be used to treat Type 1 diabetes, Type 2 diabetes and obesity. FGF-21 is administered to a mammal, preferably a human, in accord with known methods, such as oral, subcutaneous, intramuscular, inhalation, pulmonary, and/or through sustained release administration. Preferably, FGF-21 is administered orally. Most preferably, FGF-21 is administered subcutaneously.

Those skilled in the art can readily optimize pharmaceutically effective dosages and administration regimens for therapeutic compositions comprising FGF-21, as determined by good medical practice and the clinical condition of the individual patient. Generally, the formulations are constructed so as to achieve a constant local concentration of about 100 times the serum level of the growth factor or 10 times the tissue concentration, as described in Buckley et al. (Proc Natl Acad Sci (USA) 82:7340-7344, 1985). Based on an FGF concentration in tissue of 5-50 ng/g wet weight, release of 50-5000 ng FGF-21 per hour is acceptable. Preferably, release of 50-4000; 50-3000; 50-2000; 50-1000; 50-500; 50-250; or 50-100 ng of FGF-21 per hour is acceptable. The appropriate dose of FGF-21 administered will result in lowering blood glucose levels and increasing energy expenditure by faster and more efficient glucose utilization.

In another aspect of the present invention, FGF-21 for use as a medicament for the treatment of Type 1 diabetes, Type 2 diabetes and obesity is contemplated.

The use of FGF-21 in the treatment of Type 2 diabetes and obesity as presented in the present invention will provide a needed therapy for these serious and debilitating disorders.

It is further considered that FGF-21 may be administered to a mammal, preferably a domestic animal, in accord with known methods, to reduce fat stores on a long term. Such treatment is of considerable economic importance to man since domestic animals supply a major portion of man's diet, and animal fat may end up as de novo fat deposits in man. The reduction of fat stores in man is of significant benefit, cosmetically and physiologically.

Having generally described the invention, the same will be more readily understood by reference to the following examples, which are provided by way of illustration and are not intended as limiting.

### EXAMPLE 1

### Expression and Purification of FGF-21 in E. coli

The bacterial expression vector pQE60 is used for bacterial expression in this example. (QIAGEN, Inc., Chatsworth, Calif.). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., and suitable single restriction enzyme cleavage sites. These elements are arranged such that a DNA fragment encoding a polypeptide can be inserted in such a way as to produce that polypeptide with the six His residues (i.e., a “6 X His tag”) covalently linked to the carboxyl terminus of that polypeptide. However, a polypeptide coding sequence can optionally be inserted such that translation of the six His codons is prevented and, therefore, a polypeptide is produced with no 6 X His tag.

The nucleic acid sequence encoding FGF-21 lacking the hydrophobic leader sequence is amplified from a cDNA clone using PCR oligonucleotide primers (based on the sequences presented in FIG. 1), which anneal to the amino terminal encoding DNA sequences of the desired portion of the FGF-21-encoding nucleic acid and to sequences in the construct 3′ to the cDNA coding sequence. Additional nucleotides containing restriction sites to facilitate cloning in the pQE60 vector are added to the 5′ and 3′ sequences, respectively.

For cloning, the 5′ and 3′ primers have nucleotides corresponding or complementary to a portion of the coding sequence of the FGF-21-encoding nucleic acid according to known method steps. One of ordinary skill in the art would appreciate, of course, that the point in a polynucleotide sequence where the 5′ primer begins can be varied to amplify a desired portion of the complete polypeptide enoding polynucleotide shorter or longer than the polynucleotide which encodes the mature form of the polypeptide.

The amplified nucleic acid fragments and the vector pQE60 are digested with appropriate restriction enzymes and the digested DNAs are then ligated together. Insertion of FGF-21-encoding DNA into the restricted pQE60 vector places the FGF-21 polypeptide coding region including its associated stop codon downstream from the IPTG-inducible promoter and in-frame with an initiating AUG codon. The associated stop codon prevents translation of the six histidine codons downstream of the insertion point.

The ligation mixture is transformed into competent E. coli cells using standard procedures such as those described in Sambrook, et al., 1989; Ausubel, 1987-1998. E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing FGF-21, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH8. The cell debris is removed by centrifugation, and the supernatant containing the LP polypeptide is dialyzed against 50 mM Na-acetate buffer pH6, supplemented with 200 mM NaCl. Alternatively, FGF-21 can be successfully refolded by dialyzing it against 500 mM NaCl, 20% glycerol, 25 mM Tris/HCl pH7.4, containing protease inhibitors.

If insoluble protein is generated, the protein is made soluble according to known method steps. After re-naturation, the FGF-21 is purified by ion exchange, hydrophobic interaction, and size exclusion chromatography. Alternatively, an affinity chromatography step such as an antibody column is used to obtain a purified form of the FGF-21. The purified polypeptide is stored at 4° C. or frozen at −40° C. to −120° C.

### EXAMPLE 2

### Tissue Distribution of FGF-21-encoding mRNA

Northern blot analysis is carried out to examine expression of FGF-21 encoding mRNA in human tissues, using methods described by, among others, Sambrook, et al., cited above. A cDNA probe preferably encoding the entire FGF-21 polypeptide is labeled with ³²P using the Rediprime™ DNA labeling system (Amersham Life Science), according to the manufacturer's instructions. After labeling, the probe is purified using a CHROMA SPIN-100™ column (Clontech Laboratories, Inc.), according to the manufacturer's protocol number PT1200-1. The purified and labeled probe is used to examine various human tissues for FGF-21 mRNA. Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Various tissues examined: 1) brain; 2)heart; 3) skeletal muscle; 4)colon; 5)thymus; 6)spleen; 7)kidney; 8)liver; 9)small intestine; 10)placenta; 11)lung; 12)PBL.

Following hybridization and washing, the blots are mounted and exposed to film at −70° C. overnight, and developed according to standard procedures.

The above technique demonstrates that FGF-21 is expressed primarily in the liver, kidney and muscle.

### EXAMPLE 3

### Glucose Uptake in 3T3-1 Adipocytes

3T3-L1 cells are obtained from the American Type Culture Collection (ATCC, Rockville, Md.). Cells are cultured in growth medium (GM) containing 10% iron-enriched fetal bovine serum in Dulbecco's modified Eagle's medium. For standard adipocyte differentiation, 2 days after cells reached confluency (referred as day 0), cells are exposed to differentiation medium (DM) containing 10% fetal bovine serum, 10 μg/ml of insulin, 1 μM dexamethasone, and 0.5 μM isobutylmethylxanthine, for 48 h. Cells then are maintained in post differentiation medium containing 10% fetal bovine serum, and 10 μg/ml of insulin.

Glucose Transport Assay—Insulin Dependent—Hexose uptake, as assayed by the accumulation of 0.1 mM 2-deoxy-D-[¹⁴C]glucose, is measured as follows: 3T3-L1 adipocytes in 12-well plates are washed twice with KRP buffer (136 mM NaCl, 4.7 mM KCl, 10 mM NaPO₄, 0.9 mM CaCl₂, 0.9 mM MgSO₄, pH 7.4) warmed to 37 ° C. and containing 0.2% BSA, incubated in Leibovitz's L-15 medium containing 0.2% BSA for 2 h at 37° C. in room air, washed twice again with KRP containing, 0.2% BSA buffer, and incubated in KRP, 0.2% BSA buffer in the absence (Me₂SO only) or presence of wortmannin for 30 min at 37 ° C. in room air. Insulin is then added to a final concentration of 100 nM for 15 min, and the uptake of 2-deoxy-D-[¹⁴C]glucose is measured for the last 4 min. Nonspecific uptake, measured in the presence of 10 μM cytochalasin B, is subtracted from all values. Protein concentrations are determined with the Pierce bicinchoninic acid assay. Uptake is measured routinely in triplicate or quadruplicate for each experiment. A concentration response of FGF-21 in 3T3-L1 adipocytes is shown in FIG. 2. The effect of acute and chronic pretreatment of 3T3-L1 adipocytes with FGF-21 in the presence of insulin is shown in FIG. 4, indicating that FGF-21 positively influences insulin-dependent glucose uptake upon 72 hour treatment.

Glucose Transport Assay—Insulin Independent—3T3-L1 fibroblast were plated in 96- well plates and differentiated into fat cells(adipocytes) for 2 weeks. After differentiation they were starved in serum-free medium and treated with FGF-21 for 24 hours. Upon treatment cells were washed twice with KRBH buffer,containing 0.1% BSA. Glucose uptake was performed in the presence of labeled glucose( without insulin) in KPBH buffer, FIG. 3.

### EXAMPLE 4

### Ex vivo Glucose Transport

Glucose Transport Assay:

Krebs-Henseleit Buffer Stock Solutions:

Stock 1: NaCl (1.16 M); KCl (0.046 M); KH₂PO₄ (0.0116 M); NaHCO₃ (0.0253 M).

Stock 2: CaCl₂ (0.025 M); MgSO₄ (2H₂O) (0.0116 M).

BSA: Use ICN Cohn Fraction V, fatty acid free BSA directly without dialysing.

Media Preparation: Add 50 ml of Krebs stock 1 to 395 ml of dH₂O and gas with 95% O₂/5% CO₂ for 1 hour. Add 50 ml of stock 2 and bring to 500 ml with dH₂O. Add 500 mg of ICN fatty acid free BSA.

Preincubation and Incubation Media: 32 mM Mannitol, 8 mM Glucose

Wash Media: 40 mM Mannitol, 2 mM Pyruvate

Transport Media: 39 mM Mannitol, 1 mM 2-DG; 32 mM Mannitol, 8 mM 3-O-MG.

Insulin Solution: (Porcine Insulin [Lilly] 100,000,000 μU/ml) at a final concentration of 2000 μU/ml or 13.3 nM.

Radioactive Label Media Preparation: Specific activities used: 2DG=1.5 mCi/ml; 3-O-MG=437 μCi/ml; or, Mannitol=8 μCi/ml

Procedure:

Rats are anesthetized with 0.1 cc Nembutal per 100 g B. W. Muscle tissue is excised and rinsed in 0.9% saline then placed in pre-incubation media (2 ml) at 29° C. for 1 hour. The muscle tissue is transferred to incubation media (2 ml; same as preinc. except including insulin or test compound) and incubated for 30 min (depends upon experimental conditions). The muscle tissue is then transferred to wash media (2 ml) for 10 min at 29° C., then transferred to label media (1.5 ml) for 10 min (3-O-MG) or 20 min (2DG). The muscle tissue is trimmed, weighed and placed in polypropylene tubes on dry ice. 1 ml of 1 N KOH is added to the tubes which are then placed in a 70° C. water bath for 10-15 min., vortexing the tubes every few minutes. The tubes are cooled on ice and 1 ml of 1 N HCl is added, then mixed well. 200 μl of supernatant is then put in duplicate scintillation vials and counted on a scintillation counter compared to known radioactive standards.

Contraction:

For contraction, the muscles are first incubated for 1 hour in preincubation/incubation media. After 1 hour, one muscle of each pair (one pair per rat) is pinned to the stimulation apparatus and the other muscle is transferred to a new flask of incubation media. The contracted muscle is stimulated by 200 msec trains of 70 Hz with each impulse in a train being 0.1 msec. The trains are delivered at 1/sec at 10-15V for 2×10 min with a 1 minute rest in between. At the end of the stimulation period, the muscle is removed from the stimulation apparatus and placed in wash media for 10 min, followed by label media as outlined above.

### EXAMPLE 5

### Ob/ob Obesity Model

A study in an obesity model using male ob/ob mice was done to monitor plasma glucose levels after treatment with FGF-21 compared to vehicle and insulin control groups. The test groups of male ob/ob mice (7 weeks old) were injected with vehicle alone (PBS), insulin (4 U/day), or FGF-21 (5 μg/day and 25 μg/day), subcutaneously (0.1 ml, b.i.d) for seven days. Blood was collected by tail clip bleeding on days 1, 3, and 7, one hour after the first compound injection and plasma glucose levels were measured using a standard protocol. The results of the study are shown in Table 1. FGF-21 (25 μg/day) significantly reduced plasma glucose levels on days 3 and 7 when compared to the vehicle control group. Thus, FGF-21 stimulates glucose uptake in this mouse obesity model.

TABLE 1

| | Day 1 | | Day 3 | | Day 7 | |
|---|---|---|---|---|---|---|
| | Plasma | | Plasma | | Plasma | |
| Test | glucose | | glucose | | glucose | |
| Groups | (mg/dl) | SEM | (mg/dl) | SEM | (mg/dl) | SEM |
| Vehicle | 410 | 28 | 443 | 31 | 482 | 28 |
| Insulin 4U/d | 410 | 27 | 362 | 12 | 304 | 9 |
| FGF-21 5 μg/d | 408 | 29 | 423 | 28 | 418 | 30 |
| FGF-21 25 μg/d | 402 | 13 | 354 | 37 | 340 | 34 |

### EXAMPLE 6

### ZDF Rat Study

ZDF male rats ( diabetic, fat rats; 8 weeks of age at beginning of study, Charles River-GMI). Rats are fed Purina 5008 feed ad libitum. The following test groups are set up: Saline; Insulin4 U/day; FGF-21, 500 ug/day Acute (Acute dosing group is dosed once and bled at T=0, 2, 4, 8, and 24 hours post dose); FGF-21, 100 ug/day; FGF-21, 250 ug/day; FGF-21, 500 ug/day; FGF-21(once/day) 500 ug/ml; Lean Saline; Lean Insulin 4U/day; Lean FGF-21 500ug/day (Lean groups represent non-diabetic, lean, ZDF rats).

Compounds are injected s.c. (b.i.d.), except for the second 500 ug/day group which receives one injection per day for the duration of the study (7 days). Control rats are injected with vehicle (PBS; 0.1 ml).

Following 7 days of dosing, the animals are subjected to an oral glucose tolerance test. Blood for glucose and triglycerides are collected by tail clip bleeding without anesthetic. The effects of FGF21 on plasma glucose levels in male diabetic, ZDF rats is indicated in Table 2. The administration of FGF-21 reduces plasma glucose levels in a dose-dependent manner.

TABLE 2

| | | Humulin | FGF-21 | FGF-21 | FGF-21 |
|---|---|---|---|---|---|
| | Vehicle | (4U) | (20 ug) | (200 ug) | (2000 ug) |
| Day -1 | 360 | 355 | 365 | 362 | 368 |
| Day 3 | 375 | 240 | 340 | 220 | 210 |
| Day 7 | 490 | 235 | 400 | 218 | 225 |

(plasma glucose levels are measured in mg/dl)

Furthermore, FGF-21 did not induce hypoglycemia in lean ZDF rats when compared to rats does with insulin, Table 3. This data indicates that FGF-21 affects plasma glucose levels in an insulin independent manner, and thus is also useful in the treatment of Type 1 diabetes.

TABLE 3

| | Vehicle | Humulin (4U) | FGF-21 (500 ug) |
|---|---|---|---|
| Day 3 | 134 | 42 | 139 |
| Day 7 | 137 | 47 | 134 |

(glucose levels are measured in mg/dl)

1-25. cancelled.

26. A method of treating a mammal exhibiting Type 2 diabetes said method comprising administering to said mammal a composition comprising a therapeutically effective amount of FGF-21 which has at least about 95% amino acid sequence identity to the amino acid sequence shown in SEQ ID NO:2.

27. The method of claim 26 wherein said mammal is a human subject and said treatment is sufficient to achieve at least one of the following modifications: decrease in insulin resistance, reduction of hyperinsulinemia, increase in glucose tolerance, and reduction of hyperglycemia.

28. The method according to claim 26, wherein said FGF-21 has the amino acid sequence shown in SEQ ID NO:2.

29. A method of treating a mammal for obesity, said method comprising administering to said mammal a composition comprising a therapeutically effective amount of FGF-21 which has at least about 95% amino acid sequence identity to the amino acid sequence shown in SEQ ID NO:2.

30. The method of claim 29 wherein said mammal is a human subject which exhibits obesity and said treatment is sufficient to achieve at least one of the following modifications: reduction in body fat stores and reduction of hyperglycemia.

31. The method according to claim 29, wherein said FGF-21 has the amino acid sequence shown in SEQ ID NO:2.

32. The method of claim 29 wherein said mammal is a domestic animal and the treatment results a reduction in body fat stores.

33. A method of inducing an increase in glucose uptake in adipocye cells, said method comprising administering FGF-21 to said cells in an amount effective to induce an increase in glucose uptake.

34. The method of claim 33, wherein said increase in glucose uptake increases energy expenditure by faster and more efficient glucose utilization.

35. A method of treating a mammal exhibiting Type 1 diabetes said method comprising administering to said mammal a composition comprising a therapeutically effective amount of FGF-21 which has at least about 95% amino acid sequence identity to the amino acid sequence shown in SEQ ID NO:2.

36. The method according to claim 35, wherein said FGF-21 has the amino acid sequence shown in SEQ ID NO:2.

37. The method of claim 26, wherein said FGF-21 is administered as a protein.

38. The method of claim 29, wherein said FGF-21 is administered as a protein.

39. The method of claim 33, wherein said FGF-21 is administered as a protein.

40. The method of claim 35, wherein said FGF-21 is administered as a protein.

41. The method of claim 26, wherein said composition further comprises a pharmaceutical acceptable carrier.

42. The method of claim 29, wherein said composition further comprises a pharmaceutical acceptable carrier.

43. The method of claim 33, wherein said composition further comprises a pharmaceutical acceptable carrier.

44. The method of claim 35, wherein said composition further comprises a pharmaceutical acceptable carrier.
